(19) United States
(12) Patent Application Publication
(10) Pub. No.: US 2007/0128143 A1
(43) Pub. Date: Jun. 7, 2007

(54) Polyether-modified polysiloxanes with block character and use thereof for producing cosmetic formulations

(75) Inventors: Burghard Gruning, Essen, DE (US); Wilfried Knott, Essen, DE (US); Holger Leidreiter, Hattingen, DE (US); Jurgen Meyer, Munster, DE (US)

Correspondence Address: Leopold Presser, Scully, Scott, Murphy & Presser, 400 Garden City Plaza, Garden City, NY, 11530, US

(73) Assignee: Goldschmidt GmbH, Essen (DE)

(21) Appl. No.: 11/633,378

(22) Filed: Dec. 4, 2006

(30) Foreign Application Priority Data
Dec. 3, 2005 (DE) 10 2005 057 857.8

(51) Int. Cl. A61K 8/89; C08G 77/38

(52) U.S. Cl. 424/70.12; 525/476

(57) ABSTRACT

The invention provides a method of producing organomodified siloxanes with domain-type distribution obtained by partial or complete reaction of
A) hydrogensiloxanes with a degree of distribution (persistency ratio) (η) of components [A] and [B] in the copolymer [AB]

$$η = \frac{[ A ] [ B ]}{[ AB ]}$$

of η>1, preferably >1.1, in particular ≧1.2, with B) olefinically and/or acetylenically unsaturated compounds, the compounds resulting therefrom and their use.

### FIELD OF THE INVENTION

The invention relates to organomodified polysiloxanes with block character produced by hydrosilylation and to their use for producing cosmetic and pharmaceutical formulations.

### BACKGROUND OF THE INVENTION

A large part of cosmetic and pharmaceutical formulations consists of emulsions. The majority of these emulsions is usually of the oil-in-water type, i.e., the oil phase (“disperse phase”) is very finely distributed in the form of small droplets in the water phase (“coherent phase”). In addition, water-in-oil emulsions are also known which are characterized in that water drops are present in dispersed form in a continuous oil phase. These water-in-oil emulsions exhibit a number of application advantages. Thus, for example, they enable an occlusive film to form on the surface of the skin, which prevents the skin from drying out. At the same time, water-in-oil emulsions are known for their excellent water resistance.

Cosmetic emulsions comprise, irrespective of the type of emulsion, on average 20 to 30% oil phase for reasons of skin feel. In cases of water-in-oil emulsions, this means that, on account of the high internal phase content, extremely high stabilization of the water drops against coalescence is required.

It is known that this stabilization can take place, for example, in an excellent manner by alkyl polyethersiloxanes with a comb-like structure, as are described in EP-A-0 176 884.

The use of terminally modified polyethersiloxanes in cosmetic emulsions is also widespread. Besides their emulsion-stabilizing effect, these compounds are characterized by a velvety-silky skin feel which they impart to cosmetic emulsions.

Such an oil-in-water emulsifier is described, for example, in EP-A-1 125 574.

Another prominent substance class of the organomodified siloxanes for cosmetic applications is based on the pure alkyl-group-modified siloxanes or silicone waxes which are used, for example, for improving the spreading of cosmetic oils, or alternatively as wetting dispersion additives for producing color-improved pigment-containing formulations.

Finally, the use of siloxanes modified with cationic organo groups for haircare, as described, for example, in EP-B-0 294 642, also belongs to the prior art.

The continually growing demands on cosmetic formulations, for example in the direction of greater formulation flexibility or with regard to more elegant sensory profiles cannot be improved further using the described siloxane derivatives from the prior art. There is thus a need for new types of siloxanes with whose help further improved formulation properties can be established in a targeted way.

### SUMMARY OF THE INVENTION

According to a proposal (file reference 10 2005 001 039.3) which is hitherto still not published and to which reference is made in its entirety with regard to the present invention, a method for the targeted production of domain-type polydimethylsiloxane-poly(methylhydrogen)siloxane copolymers interspersed with SiH functions is described.

Here, mixtures consisting of methylhydrogenpolysiloxane, hexamethyldisiloxane and siloxane cycles are brought into contact over a macro-crosslinked cation exchange resin containing sulfonic acid groups at a temperature of from 10° C. to 120° C., and the resulting equilibrated organosiloxanes are isolated. The cation exchange resin used is characterized in that its product P of its specific surface area and of its average pore diameter is P<2.2×10⁻³ m³/kg, in particular <1.5×10⁻³ m³/kg, particularly preferably <1×10⁻³ m³/kg, and the specific surface area A is <50 m²/g, in particular A<35 m²/g and particularly preferably A<25 m²/g.

The ion exchange phase characterized in this way ensures, depending on the design, the partial or predominant presence of methylhydrogensiloxane domains in the resulting polydimethylsiloxane-poly(methylhydrogen-)siloxane copolymers.

The definition of domains is used as far as content is concerned in agreement with the definition of “persistence ratio” in the publication by P. Cancouet et al. “Functional Polysiloxanes, I. Microstructure of Poly(hydrogenmethylsiloxane-co-dimethylsiloxane)s Obtained by Cationic Copolymerization”, J. of Polymer Science, Par A: Polymer Chemistry, Vol 38, 826-836 (2000). According to this, persistency ratio (11) is a measure of the degree of distribution of components [A] and [B] in the copolymer [AB]:

$$η \begin{matrix}= \frac{{\overline{L}}_{exp}}{{\overline{L}}_{st}} \\ = \frac{[ A ] [ B ]}{[ AB ]}\end{matrix}$$

According to this, the persistency ratio Ti characterizes the ratio of experimentally determined block length of one monomer sequence in a copolymer to the statistically expected block length in the case of complete distribution.

Accordingly, components [A] and [B] tend toward an alternating distribution when Ti is <1, to a regular distribution when T=1 and to a domain-type distribution when η is >1. Analytically, the cumulation of SiH functionalities can be demonstrated by evaluating high-resolution 29Si-NMR spectra. The equilibration products used according to the invention according to the application with the file reference: 10 2005 001 039.3 have η values >1.

The content of the above publication is hereby incorporated as reference and serves as part of the disclosure content of the present invention.

These products are especially noteworthy therefore since it was not possible using the known prior art equilibration catalysts to produce equilibrates with a block-type structure, i.e., with locally increased functionalization densities (SiH clusters) along the siloxane structure, in a targeted and reproducible way.

In a second reaction stage, the hydrogensiloxanes with a domain-type structure produced in this way can be reacted with hydrocarbons having multiple bonds and/or unsaturated polyoxyalkyl ethers.

This second stage of the production can take place here in accordance with the known hydrosilylation processes, as described, for example, in U.S. Pat. Nos. 3,234,252, 4,047,958, and 3,427,271.

However, first application tests on block-type polyethersiloxanes produced in this way for technical applications such as, for example, for stabilizing flexible polyurethane foams, showed that the performance of these polyethersiloxanes with a domain-type structure was considerably worse than conventionally produced random polyethersiloxanes.

Even more surprisingly, it has now been found that the reaction products of the domain-type structured hydrogensiloxanes produced in the first stage with hydrocarbons having multiple bonds and/or unsaturated polyoxyalkyl ethers produce interface-active products with new kinds of excellent application properties in cosmetic and pharmaceutical formulations.

When using alkyl polyethersiloxanes with a block-type structure as emulsifiers for water-in-oil emulsions, it was entirely surprising to find that it is possible to achieve a similarly high emulsion-stabilizing effect as through the use of randomly uniform alkyl polyethersiloxanes.

An advantage of using polyethersiloxanes with a block-type structure, moreover, is the lower emulsion viscosity. This lower emulsion viscosity offers the advantage of better spreadability of the formulation on the skin, improved absorption behavior, and a generally somewhat lighter skin feel. Here, the alkylpolyethersiloxanes with a block-type structure generally appear to impart a more marked velvety-silky skin feel than is known from the randomly constructed alkylpolyethersiloxanes.

Surprisingly, it has also been found that the block-type modified alkylpolyethersiloxanes, especially when using high fractions of silicone oils (e.g., cyclopentasiloxanes, dimethicones) in the oil phase, are able to convey a similarly velvety-silky skin feel as was previously known only from terminally modified polyethersiloxanes, which are conventionally used as emulsifiers for stabilizing such water-in-silicone emulsions. At the same time, the alkylpolyethersiloxanes with a domain-type structure, however, exhibit a clearly better emulsion stabilization than do these.

Quite generally, it may be stated that the velvety-silky skin feel, which in particular is a feature of organosiloxanes with relatively long unmodified siloxane chains, can, with the help of these novel organosiloxanes with a domain-type structure, be combined significantly more easily with the functionalities required for the particular intended use than was possible for the hitherto obtainable randomly distributed organosiloxanes.

Thus, both emulsifiers with a domain-type structure and based on alkyl polyethersiloxanes, and also performance improvers based on alkylsiloxanes exhibited an improved skin feel. Haircare additives based on cationically modified domain-type organosiloxanes are characterized by a softer hand.

### DETAILED DESCRIPTION OF THE INVENTION

The invention therefore provides a method of producing organomodified siloxanes with domain-type distribution obtained by partial or complete reaction of
1. hydrogensiloxanes with a degree of distribution (persistency ratio) (η) of components [A] and [B] in the copolymer [AB]:

$$η = \frac{[ A ] [ B ]}{[ AB ]}$$

of η>1, preferably >1.1, in particular ≧1.2, with
2. olefinically and/or acetylenically unsaturated compounds.

The invention further provides a method of producing organomodified siloxanes, wherein, for producing the methylhydrogensiloxanes, a mixture of hexamethyldisiloxane, poly(methyl)hydrogensiloxane and siloxane cycles are equilibrated as starting material.

The invention further provides a method of producing organomodified siloxanes, wherein the olefinically unsaturated compounds of the general formula (1) CH₂═CHR—(CH₂)ₙ-A are used, in which A is at least one radical selected from the following:
1. optionally substituted linear and/or cyclic hydrocarbons which may optionally contain functional groups,
2. randomly or blockwise constructed oxyalkylene chains —(OAlk)ₛ-R,
   in which
   R is H, a C₁₋₁₈-alkyl radical which is optionally substituted and/or optionally contains heteroatoms, or the radical of a monobasic and/or polybasic C₁₋₂₂-carboxylic acid, which can optionally contain hydroxyl groups, the radical of an inorganic acid selected from the group sulfuric acid, sulfonic acid, phosphoric acid,
   OAlk is the radical -(EO)_{b}—(PO)_{c}—(BO)_{d}-(DO)ₑ—(SO)_{f}— where
      EO is ethylene oxide radical,
      PO is propylene oxide radical,
      BO is butylene oxide radical,
      DO is dodecenyl oxide radical,
      SO is optionally alkyl-substituted styrene oxide radical,
      b, c, d, e, f are ≧0, in particular b is 0 to 50, c is 0 to 50,
      d, e, f are identical or different and are 0 to 10 and the sum
      b+c+d+e+f is a and
      a is 1 to 50, preferably 1 to 30, in particular 3 to 20,
         and
3. radicals containing oxirane groups of the formulae 3a to 3d 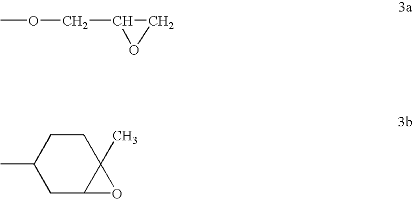
(and when x=0) 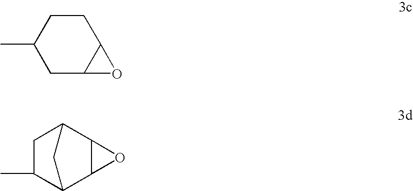
4. at least one amine/ammonium radical, -MZ, -[M-Z]^{g+}h*X^{j-}, in which
   M is a divalent radical chosen from the group 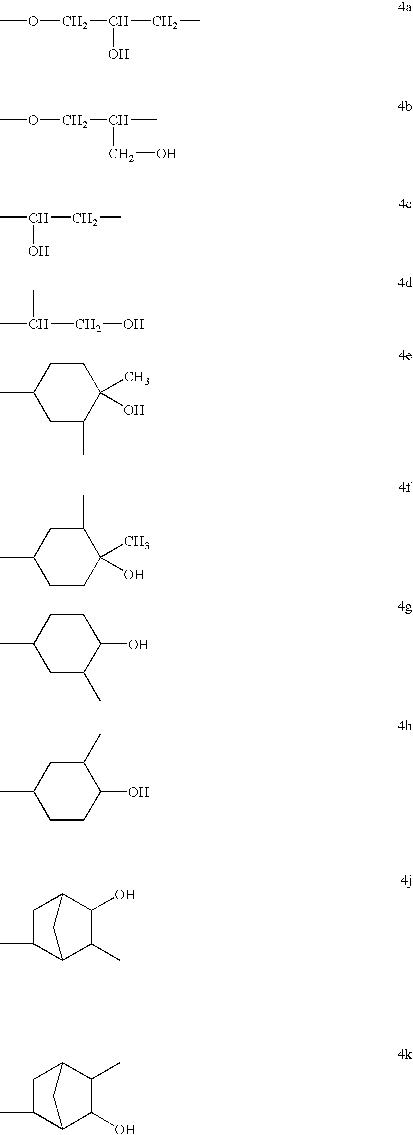
   and Z may be one of the radicals 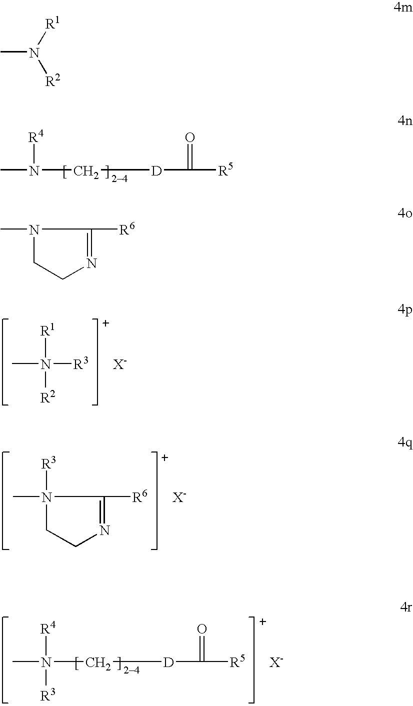
in which
   R¹, R², R³, R⁴, independently of one another, are H, C₁₋₂₂-alkyl radicals which may also contain hydroxyl groups,
      R⁵, R⁶ are C₁₋₂₂-alkyl radicals which may also contain hydroxyl groups,
      D is —O− or —NR⁷— where R⁷=alkyl or hydroxyalkyl radical with 1-4 carbon atoms,
   X⁻ may be an organic or inorganic anion,
   h*j is the same as the numerical value of g;
5. polyhydroxyorganyl radicals of the general formula —R⁸—PH, where the radical
   R⁸ acts as spacer between siloxane backbone and polyhydroxyorganyl radical or sugar radical and is of the type known from the prior art for polyhydroxyorganyl- or sugar-modified siloxanes,
   PH is a polyhydroxyorganyl radical which contains a defined number n of (C—OH) groups, where n is ≧2, preferably 5 to 15,
      from the group mono-, di-, oligo- or polysaccharide, their glycosides or corresponding derivatives, in particular glucose, maltose, raffinose, sorbitol, glucosamine, glucopyranosylamine, glucamine, N-methylglucamine, isomaltamine, gluconic acid, heptagluconic acid; 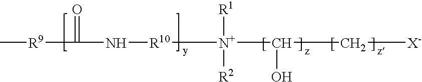
6. betaine groups,
   in which
      X is —COO—, SO₃⁻, PO₄²⁻,
      R⁹ is alkylene radicals having up to 10 carbon atoms,
      R¹⁰ is alkylene radicals having up to 2 to 6 carbon atoms,
      y is 0 or 1,
      Z is 0 or 1,
      z′ is 1, 2 or 3,
      and
      R¹, R² are identical or different and have the meaning given above, together form an imidazoline ring or are —CH₂—CH₂—OH, —(CH₂)_{z}—X⁻,
7. guanidine groups of the formulae (6a, 6b or 6c)
   R¹¹=—N-G (6a)
   R¹¹=—N-Q⁺A⁻ (6b)
   R¹¹=—(N)ₓ—S (6c)
in which
   G is a guanidino group with the general formula (6a′, 6a²) 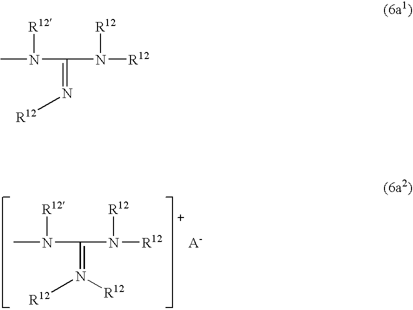
   and/or salts or hydrates thereof, in which
   R¹² independently of the others, is hydrogen or an optionally branched hydrocarbon radical optionally containing double bonds, or
   R^{12′} may be R¹² or an alkylene group which is joined to M via carbon atoms or heteroatoms and thus forms a 5- to 8-membered ring and
   N is a di- or polyvalent hydrocarbon radical having at least 4 carbon atoms which has a hydroxyl group and which may be interrupted by one or more oxygen atoms or nitrogen atoms or quaternary ammonium groups or esters or amide functions,
   Q⁺ is a radical of the formula (6d) 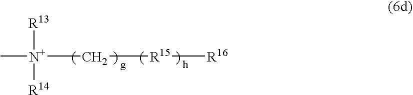
   R¹³, R¹⁴ are alkyl radicals having 1 to 4 carbon atoms,
   R¹⁵ is 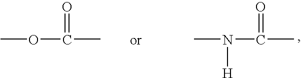
   R¹⁶ may be a monovalent hydrocarbon radical having 1 to 22 carbon atoms,
   g is 0 to 6
   h is 0 or 1,
   A⁻ is an inorganic or organic anion which originates from a customary physiologically compatible acid HA,
   S is H, a polyalkylene oxide polyether of the general formula
      CₘH₂ₘO(C₂H₄O)ₙ(C₃H₆O)ₒR¹⁷
   in which
   m is 1 to 6, in particular 3, 6,
   n, o, independently of one another, are 0 to 100, in particular 0 to 20 and the polyether has a molecular weight between 100 and 6000 g/mol and
   R¹⁷ is H or an optionally branched aromatic or alicyclic hydrocarbon radical having 2 to 30 carbon atoms, preferably 4 to 22 carbon atoms, and optionally containing double bonds, or a UV-absorbing group, in particular cinnamic acid or methoxycinnamic acid.

The compounds listed are linked to the SiH siloxanes by methods known to those skilled in the art. The compounds and the methods of producing the organomodified siloxanes are described in detail in the following U.S. Pat. Nos. 6,645,842, 4,698,178, 5,204,433, 4,891,166, 4,833,225, and 4,609,750. The contents of each of the aforementioned patents are hereby incorporated as reference and serves as part of the disclosure content of the present invention.

The invention further provides the use of the organopolysiloxanes with a domain-type structure obtained by the method according to the invention for producing cosmetic formulations. These exhibit excellent application properties.

The organosiloxanes with a block-type structure according to the invention can be used, for example, as emulsifiers or dispersion additives, as additives for an improved skin feel or an improved hand, or generally as performance improvers for example for better processing of other ingredients, depending on the type, number and distribution of the substituents.

The invention further provides the use of the organosiloxanes with a domain-type structure according to the invention in cosmetic formulations for the care and cleaning of skin and hair, in sunscreen products, in antiperspirants/deodorants, and in pigment-containing formulations from the field of decorative cosmetics.

The invention further provides the use of the compounds produced according to the invention in pharmaceutical formulations.

The invention further provides the use of the organosiloxanes with a domain-type structure according to the invention in compositions for the cleaning and care of hard surfaces, and for the cleaning and care of textiles.

Moreover, the use of the inventive compounds as coating additives in the form of, for example, substrate wetting agents, slide and flow improvers, deaerating agents, scratch resistance improvers, inter alia, is provided by the invention.

Preference is given to the use of the interface-active compounds according to the invention in cosmetic formulations for the care and cleaning of skin and hair. These may, for example, be creams or lotions for skincare, surfactant-based products for the cleaning and care of skin and hair, sunscreen products, pigment-containing products from the field of decorative cosmetics (e.g., makeup, lipsticks, powders, products for lid/eyelash coloring), products for conditioning hair, nailcare products or antiperspirants/deodorants.

In these formulations, the compounds according to the invention can be used together with the formulation constituents known for these fields of use, such as oil components, cosurfactants and coemulsifiers, consistency regulators, thickeners, waxes, UV photoprotective filters, antioxidants, hydrotropes, deodorant and antiperspirant active ingredients, active ingredients, dyes, preservatives and perfumes.

Suitable cosmetic oils are, in particular, mono- or diesters of linear and/or branched mono- and/or dicarboxylic acids having 2 to 44 carbon atoms with linear and/or branched saturated or unsaturated alcohols having 1 to 22 carbon atoms.

Likewise suitable are the esterification products of aliphatic, difunctional alcohols having 2 to 36 carbon atoms with monofunctional aliphatic carboxylic acids having 1 to 22 carbon atoms. Monoesters suitable as oil components are, for example, the methyl esters and isopropyl esters of fatty acids having 12 to 22 carbon atoms, such as, for example, methyl laurate, methyl stearate, methyl oleate, methyl erucate, isopropyl palmitate, isopropyl myristate, isopropyl stearate, isopropyl oleate. Other suitable monoesters are, for example, n-butyl stearate, n-hexyl laurate, n-decyl oleate, isooctyl stearate, isononyl palmitate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, and esters which are obtainable from technical-grade aliphatic alcohol cuts and technical-grade, aliphatic carboxylic acid mixtures, e.g., esters of unsaturated fatty alcohols having 12 to 22 carbon atoms and saturated and unsaturated fatty acids having 12 to 22 carbon atoms, as are accessible from animal and vegetable fats. However, naturally occurring monoester and wax ester mixtures as are present, for example, in jojoba oil or in sperm oil are also suitable.

Suitable dicarboxylic esters are, for example, di-n-butyl adipate, di-n-butyl sebacate, di(2-ethylhexyl)adipate, di(2-hexyldecyl)succinate, diisotridecyl azelate. Suitable diol esters are, for example, ethylene glycol dioleate, ethylene glycol diisotridecanoate, propylene glycol di(2-ethylhexanoate), butanediol diisostearate and neopentyl glycol dicaprylate.

Further fatty acid esters which can be used are the esterification products of benzoic acid with linear or branched fatty acids having 8 to 22 carbon atoms.

Suitable oil components are also dialkylcarboxylic esters which can carry linear or branched alkyl chains having 6 to 22 carbon atoms.

As an oil component, it is likewise possible to use fatty acid triglycerides, with the naturally occurring oils and fats among these being preferred. Thus, for example, natural, vegetable oils, e.g. olive oil, sunflower oil, soybean oil, peanut oil, rapeseed oil, almond oil, palm oil, but also the liquid fractions of coconut oil or of palm kernel oil, and animal oils, such as, for example, neatsfoot oil, the liquid fractions of beef tallow or else synthetic triglycerides, such as reaction products with caprylic/capric acid mixtures or with isostearic acid, triglycerides of technical-grade oleic acid or of palmitic acid/oleic acid mixtures are suitable as oil components.

In addition, hydrocarbons, in particular, liquid paraffins and isoparaffins, can be used. Examples of hydrocarbons which can be used are paraffin oil, isohexadecane, polydecene, vaseline, paraffinum perliquidum, squalane.

In addition, it is also possible to use linear or branched fatty alcohols, such as oleyl alcohol or octyldodecanol, and fatty alcohol ethers, such as dicaprylyl ether.

Suitable silicone oils and silicone waxes are, for example, polydimethylsiloxanes, cyclomethylsiloxanes, and aryl- or alkyl- or alkoxy-substituted polymethylsiloxanes not claimed according to the invention.

Furthermore, surfactants, emulsifiers or dispersion auxiliaries can additionally be used. These are preferably nonionic, anionic, cationic or amphoteric surfactants or emulsifiers.

Suitable nonionogenic emulsifiers or surfactants are compounds from at least one of the following groups:
addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group
C_{12/18} fatty acid monoesters and diesters of addition products of from 1 to 30 mol of ethylene oxide onto glycerol
glycerol mono- and diesters and sorbitan mono- and diesters of saturated and unsaturated fatty acids having 6 to 22 carbon atoms and ethylene oxide addition products thereof
alkyl mono- and oligoglycosides having 8 to 22 carbon atoms in the alkyl radical and ethylene oxide addition products thereof
addition products of from 2 to 200 mol of ethylene oxide onto castor oil and/or hydrogenated castor oil
partial esters based on linear, branched, unsaturated or saturated C₆₋₂₂ fatty acids, ricinoleic acid, and 12-hydroxystearic acid and glycerol, polyglycerol, pentaerythritol, dipentaetythritol, sugar alcohols (e.g., sorbitol), alkyl glucosides (e.g., methyl glucoside, lauryl glucoside or cetearyl glucoside), and polyglucosides (e.g., cellulose). Here, the use of partial esters of glycerol and of polyglycerol is preferred. These are, for example, glycerol oleate, glycerol isostearate, polyglycerol laurates, polyglycerol isostearates, polyglycerol oleates, polyglycerol polyricinoleates, polyglycerol poly-12-hydroxystearates, distearoyl polyglyceryl-3 dimer dilinoleate or polyglyceryl-4 diisostearate polyhydroxystearate sebacate
mono-, di- and trialkyl phosphates, and mono-, di- and/or tri-PEG alkyl phosphates and salts thereof
polysiloxane-polyether copolymers (dimethicone copolyols) which do not have a domain character according to the invention, such as, for example, PEG/PPG-20/6 dimethicone, PEG/PPG-20/20 dimethicone, bis-PEG/PPG-20/20 dimethicone, PEG-12 or PEG-14 dimethicone, PEG/PPG-14/4 or 14/12 or 20/20 or 18/18 or 17/18 or 15/15. Of particular suitability here are products such as bis-PEG/PPG-14/14 dimethicone or PEG/PPG-16/16 PEG/PPG-16/16 dimethicone
polysiloxane-polyalkyl-polyether copolymers which do not have a domain character according to the invention, or corresponding derivatives, such as, for example, lauryl or cetyl dimethicone copolyols
mixed esters of pentaerythritol, fatty acids, citric acid and fatty alcohol as in DE-B-1 165 574 and/or mixed esters of fatty acids having 6 to 22 carbon atoms, methylglucose and polyols, preferably glycerol or polyglycerol.

Anionic emulsifiers or surfactants can also additionally be used.

These contain hydrophilic anionic groups, such as, for example, carboxylate, sulfate, sulfonate or phosphate groups and a lipophilic radical. Skin-compatible anionic surfactants are known to those skilled in the art in large numbers and are commercially available. These are, in particular, alkyl sulfates or alkyl phosphates in the form of their alkali metal, ammonium or alkanolammonium salts, alkyl ether sulfates, alkyl ether carboxylates, acyl sarcosinates, and sulfosuccinates and acyl glutamates in the form of their alkali metal or ammonium salts. Suitable anionic emulsifiers are also neutralized or partially neutralized citric acid esters, such as, for example, glyceryl stearate citrate, or partially saponified glyceryl stearate (glyceryl stearate SE).

Cationic emulsifiers and surfactants can also be added.

Quaternary ammonium compounds in particular can be used as such, for example, alkyltrimethylammonium halides, such as, for example, cetyltrimethylammonium chloride or bromide or behenyltrimethylammonium chloride, but also dialkyldimethylammonium halides, such as, for example, distearyldimethylammonium chloride. In addition, monoalkylamidoquats, such as, for example, palmitamidopropyltrimethylammonium chloride or corresponding dialkylamidoquats can be used. It is also possible to use readily biodegradable quaternary ester compounds, which are mostly quaternized fatty acid esters based on mono-, di- or triethanolamine. In addition, alkylguanidinium salts can be added as cationic emulsifiers.

It is also possible to use amphoteric surfactants, such as, for example, betaines, amphoacetates or amphopropionates together with the polyglycerol esters according to the invention.

In addition, known stabilizers and thickeners for oil and water phases can be used.

For thickening oil phases, all thickeners known to one skilled in the art are suitable. In particular, mention may be made here of waxes, such as hydrogenated castor wax, beeswax or microwax. In addition, it is also possible to use inorganic thickeners, such as silica, alumina or sheet silicates (e.g., hectorite, laponite, saponite). Preferably, these inorganic oil phase thickeners are hydrophobically modified.

Suitable consistency regulators for oil-in-water emulsions are primarily fatty alcohols or hydroxy fatty alcohols having 12 to 22 and preferably 16 to 18 carbon atoms and also partial glycerides, fatty acids or hydroxy fatty acids.

Suitable thickeners for water phases are, for example, polysaccharides, in particular xanthan gum, guar and guar derivatives, agar agar, alginates and tyloses, cellulose and cellulose derivatives, such as, for example, carboxymethylcellulose, hydroxyethylcellulose, hydroxymethylpropylcellulose, also alkyl-modified sugar derivatives, such as, for example, cetylhydroxyethylcellulose, also higher molecular weight polyethylene glycol mono- and diesters of fatty acids, carbomers (crosslinked polyacrylates), polyacrylamides, polyvinyl alcohol and polyvinylpyrrolidone, surfactants, such as, for example, ethoxylated fatty acid glycerides, esters of fatty acids with polyols, such as, for example, pentaerythritol or trimethylolpropane, fatty alcohol ethoxylates with a narrowed homolog distribution or alkyl oligoglucosides.

Active ingredients are understood as meaning, for example, tocopherol, tocopherol acetate, tocopherol palmitate, ascorbic acid, deoxyribonucleic acid, coenzyme Q10, retinol derivatives and retinyl derivatives, bisabolol, allantoin, phytantriol, panthenol, AHA acids, amino acids, hyaluronic acid, creatin (and creatin derivatives), guanidine (and guanidine derivatives), ceramides, phyto sphingo sine (and phytosphingosine derivatives), sphingo sine (and sphingosine derivatives), pseudoceramides, essential oils, peptides, protein hydrolysates, plant extracts and vitamin complexes.

Suitable UV filters, self-tanning agents, preservatives, antioxidants, hydrotropes, antiperspirant active ingredient, deodorants, dyes, perfume oils, insect repellents, as are described in the prior art, can also be used.

The following examples are provided for illustrative purposes and in no ways are intended to limit the scope of the present invention.

### EXAMPLE 1 (NOT ACCORDING TO THE INVENTION)

Preparation of a Polydimethylsiloxane-poly(methylhydrogen)siloxane Copolymer Interspersed with SiH Functions in a Domain-Type Manner

In a 2 l four-neck round-bottomed flask with KPG stirrer, reflux condenser and nitrogen blanketing, 16.3 g of hexamethyldisiloxane were admixed with 256.6 g of a poly(methylhydrogen)siloxane (molar mass: 2868.1 g/mol, SiH value: 15.69 Val/kg) and 867.7 g of decamethylcyclopentasiloxane and with 68.5 g of a predried Purolite C 150 MBH (crosslinked, macroporous sulfonic acid polystyrene resin), and the reaction mixture was heated at 60° C. for 6 hours with stirring. The sulfonic acid solid-phase catalyst was removed by filtration after cooling the reaction matrix.

### EXAMPLE 2 (ACCORDING TO THE INVENTION)

Preparation of an Alkyl Polyethersiloxane with a Domain-Type Structure:

In a 250 ml four-neck flask fitted with KPG stirrer, reflux condenser and nitrogen blanketing, 100 g of the polydimethylsiloxane-poly(methylhydrogen)siloxane copolymer with a domain-type structure (SiH value: 3.53 Val/kg) were heated to 90° C. and admixed with 10 ppm of platinum (based on the total mixture in the form of cis-diamminoplatinum(II) chloride). 30 g of hexadecene-1 were added, whereupon the hydrosilylation reaction started. After about 30 minutes, the reaction mixture was admixed with 47.7 g of a hydroxyfunctional allyl polyether constructed from ethylene oxide units which had an average molecular weight of about 500 g/mol. One hour after the addition of reactants was complete, a further 33.3 g of hexadecene-1 were added and the mixture was left for a further 2 hours at the reaction temperature. Gas-volumetric SiH determination (decomposition of an aliquot sample amount with sodium butoxide solution in a gas burette) ensured a quantitative conversion. The virtually colorless, but cloudy alkylpolyethersiloxane could be used directly as an emulsifier.

### EXAMPLE 3 (NOT ACCORDING TO THE INVENTION)

Analogously to example 2, 100 g of a randomly equally distributed polydimethylsiloxane-poly(methylhydrogen)siloxane copolymer (SiH value: 3.53 Val/kg) were heated to 90° C. and admixed with 10 ppm of platinum (based on the total mixture in the form of cis-diamminoplatinum(II) chloride). Following the addition of 30 g of hexadecene-1, the mixture was stirred for half an hour and the reaction mixture was supplemented through the addition of 47.7 g of a hydroxyfunctional allyl polyether constructed from ethylene oxide units (average molecular weight of about 500 g/mol). After one hour, a further 33.3 g of hexadecene-1 were added and after-reacted for 2 hours. Gas-volumetric SiH determination (decomposition of an aliquot sample amount with sodium butoxide solution in a gas burette) demonstrated a quantitative conversion. The virtually colorless, clear alkylpolyethersiloxane could be used directly as an emulsifier.

### EXAMPLE 4 (ACCORDING TO THE INVENTION)

Preparation of an Alkylsiloxane with a Domain-Type Structure and Ammonium-Functional Groups

In a 250 ml four-neck flask fitted with KPG stirrer, reflux condenser and nitrogen blanketing, 100 g of the polydimethylsiloxane-poly(methylhydrogen)siloxane copolymer with a domain-type structure obtained as in example 1 (SiH value: 3.53 Val/kg) were heated to 90° C. and admixed with 10 ppm of platinum (based on the total mixture in the form of cis-diamminoplatinum(II) chloride). 55.5 g of hexadecene-1 were added, whereupon the hydrosilylation reaction started. After about 30 minutes, 15.7 g of allyl glycidyl ether were added. The mixture was left for 2 hours at the reaction temperature and then a gas-volumetric SiH determination (decomposition of an aliquot sample amount with sodium butoxide solution in a gas burette) was carried out, which confirmed a quantitative conversion. The reflux condenser was replaced with a distillation bridge and excess allyl glycidyl ether as well as a small amount of siloxane cycles were distilled off at reduced pressure (30 mPas). The epoxy oxygen content in the cooled reaction mixture was 1%.

In the next step, 167.6 g of the epoxy-modified alkylsiloxane were added dropwise to a mixture consisting of 31.6 g of coconut fatty acid amide amine (amid CNF/Degussa) and 6.6 g of acetic acid, and 50 g of isopropanol with stirring at 25° C. in a 500 ml four-neck flask. The mixture was heated at the reflux temperature for 6 hours and then the clear flask contents were left to cool.

### APPLICATION EXAMPLES

The application examples below are intended to illustrate the subject matter of the invention in more detail without limiting it to these examples. The concentration data in all of the examples is given as % by weight.

### Application Example 1

This application example is intended to show that a typical comb-type alkylpolyethersiloxane with a domain-type structure and constructed according to the invention (synthesis example 1) displays excellent application properties as water-in-oil emulsifier, which have additional advantages over the already excellent application properties of water-in-oil emulsifiers based on randomly constructed, comb-type alkylpolyethersiloxanes.

| | | Emulsions | | | |
|---|---|---|---|---|---|
| | | 1 | C1 | 2 | C2 |
| A | Alkylpolyethersiloxane | 2.00% | | 2.00% | |
| | with a domain-type | | | | |
| | structure from | | | | |
| | synthesis example 2 | | | | |
| | Randomly constructed | | 2.00% | | 2.00% |
| | alkylpolyethersiloxane | | | | |
| | from comp. Ex. 3 | | | | |
| | Hydrogenated castor oil | 0.10% | 0.10% | | 0.10% |
| | Microcrystalline wax | 0.10% | 0.10% | | 0.10% |
| | Caprylic capric triglyceride | 8.90% | 8.90% | | |
| | Ethylhexyl palmitate | 8.90% | 8.90% | 6.00% | 6.00% |
| | Paraffinum perliquidum | | | 11.80% | 11.80% |
| B | Glycerol | 3.10% | 3.10% | 3.10% | 3.10% |
| | NaCl | 0.80% | 0.80% | 0.80% | 0.80% |
| | Bronopol | 0.05% | 0.05% | 0.05% | 0.05% |
| | Water | ad 100 | ad 100 | ad 100 | ad 100 |
| | Stability | stable | stable | stable | stable |
| | Viscosity* [Pas] | 36 | 45 | 31 | 37 |
| | Application properties | very | good | very | good |
| | | good | | good | |

*Brookfield RVT spindle C, 10 rpm

The water-in-oil emulsions 1 and 2 according to the invention exhibited stabilities which are absolutely comparable with those known from randomly modified alkylpolyethersiloxanes. Moreover, in the example emulsions 1 and 2 according to the invention, however, a lower emulsion viscosity was observed.

A lower emulsion viscosity for a constant stability is advantageous in the case of W/O inasmuch as it allows better spreadability of the emulsion on the skin.

Additionally, the emulsions according to the invention exhibited a generally somewhat lighter skin feel than was the case with the comparison emulsions based on randomly modified alkylpolyethersiloxanes.

Thus, the block-type alkylpolyethersiloxanes according to the invention are characterized by surprisingly positive application properties.

### Application Example 2

For the formulation of water-in-silicone emulsions, as are used primarily for example in the areas of antiperspirants/deodorants or in the field of decorative cosmetics, previously mostly polyethersiloxanes with a linearly constructed α-ω structure have been used in practice. The reason for this is the excellent compatibility of the unmodified siloxane backbone of such a molecule with the silicone oil phase.

Although such emulsifiers are characterized by a very pleasant, velvety-silky skin feel, their stabilization potential is limited.

Comb-type alkylpolyethersiloxanes with a domain-type structure according to the invention as from synthesis example 1 now lead, especially in the field of W/Si emulsions, to a similarly pleasant, velvety-silky skin feel, but exhibit significantly improved stability (as can be seen from the comparison table).

The comparative example chosen is a customary W/Si emulsifier with α-ω modification (bis-PEG/PPG-14/14 dimethicone).

| | | Emulsions | | | |
|---|---|---|---|---|---|
| | | 1 | C1 | 2 | C2 |
| A | Alkylpolyethersiloxane | 2.00% | | 1.00% | |
| | with a domain-type | | | | |
| | structure from | | | | |
| | synthesis example 2 | | | | |
| | Bis-PEG/PPG-14/14 | | 2.00% | | 1.00% |
| | dimethicone | | | | |
| | Cyclopentasiloxane | 23.00% | 23.00% | 24.00% | 24.00% |
| B | NaCl | 0.60% | 0.60% | 0.60% | 0.60% |
| | Bronopol | 0.05% | 0.05% | 0.05% | 0.05% |
| | Water | ad 100 | ad 100 | ad 100 | ad 100 |
| | Stability | stable | moderate¹⁾ | stable | poor²⁾ |
| | Viscosity* [Pas] | 23 | 26 | 20 | 31 |
| | Application properties | very | very | very | very |
| | | good | good | good | good |

*Brookfield RVT spindle C, 10 rpm
¹⁾Significant water separation after three freeze-thaw cycles (3× −15° C./20° C.)
²⁾Emulsion decomposition after three thaw cycles (3× −15° C./20° C.); significant water separation after storage for one month at 45° C.

The investigated emulsions are extremely critical, wax-free formulations. The emulsions with a reduced emulsifier content of 1% in particular disclose the enormously strong stabilization effect of the polyethersiloxanes according to the invention.

### Application Example 3

This application example is intended to show that a typical comb-type alkylpolyethersiloxane with a domain-type structure constructed according to the invention (synthesis example 2) displays excellent application properties as ingredient in skin-cleaning compositions, which has additional advantages over the already excellent application properties of known refatting agents based on randomly constructed, comb-type alkylpolyethersiloxanes.

| | Skin-cleaning composition | | | |
|---|---|---|---|---|
| | 1 | C1 | C2 | C3 |
| Sodium laureth sulfate 28% | 32.0% | 32.0% | 32.0% | 32.0% |
| Perfume | 0.5% | 0.5% | 0.5% | 0.5% |
| Alkylpolyethersiloxane with a domain-type | 0.5% | — | — | — |
| structure from synthesis example 2 | | | | |
| Randomly constructed alkylpolyethersiloxane | — | 0.5% | — | — |
| from comp. Ex. 3 ABIL ® EM 90 Cetyl | | | | |
| PEG/PPG-10/1 Dimethicone | | | | |
| ABIL ® B 88184 PEG/PPG-20/6 dimethicone | — | — | 0.5% | — |
| ABIL ® B 8832 Bis-PEG/PPG-20/20 dimethicone | — | — | — | 0.5% |
| REWOTERIC ® AM C Sodium cocoamphoacetate 30% | 6.0% | 6.0% | 6.0% | 6.0% |
| TEGO betaine F 50 | 6.0% | 6.0% | 6.0% | 6.0% |
| Citric acid (30% in water) | 1.5% | 1.5% | 1.5% | 1.5% |
| ANTIL ® 200 PEG-200 hydrogenated glyceryl | 1.5% | 1.5% | 1.5% | 1.5% |
| palmate; PEG-7 glyceryl cocoate | | | | |
| Sodium chloride | 1% | 1% | 1% | 1% |
| Water | ad 100 | ad 100 | ad 100 | ad 100 |
| Evaluation: | | | | |
| Skin feel | 5 | 4.2 | 3.2 | 4.5 |

Compared to comparison products C1 to C3, the skin-cleaning composition 1 according to the invention exhibits the best skin feel. This parameter evaluates skin smoothness and softness, the absence of sticky residues and the perceived skin moisture in the in-vivo experiment on human skin. Formulation C3 in which a polyethersiloxane with an undisturbed polydimethylsiloxane was used with bis-PEG/PPG-20/20 dimethicone also exhibits a weaker evaluation in skin feel than the skin-cleansing composition 1 according to the invention. The evaluation detailed in the table was carried out on the scale: 1=poor to 5=excellent.

### Application Example 4

This application example is intended to show that a typical comb-type alkylsiloxane with ammonium-functional groups and with a domain-type structure and constructed according to the invention (synthesis example 4) exhibits excellent application properties as conditioner for haircare products, which have additional advantages over the already excellent application properties of known conditioners based on modified siloxanes.

| | Hair shampoos | | | |
|---|---|---|---|---|
| | 1 | C1 | 2 | C2 |
| Sodium laureth sulfate, 28% | 32.0% | 32.0% | 32.0% | 32.0% |
| Perfume | 0.5% | 0.5% | 0.5% | 0.5% |
| Alkylsiloxane with ammonium-functional groups | 0.5% | — | 0.5% | — |
| with a domain-type structure from synthesis | | | | |
| example 4 | | | | |
| ABIL ® Quat 3272 Quaternium-80 | — | 0.5% | — | 0.5% |
| Jaguar C 162 Hydroxypropyl Guar | — | — | 0.2% | 0.2% |
| Hydroxypropyltrimonium chloride | | | | |
| REWOTERIC ® AM C Sodium cocamphoacetate 30% | 6.0% | 6.0% | 6.0% | 6.0% |
| TEGO betaine F 50 | 6.0% | 6.0% | 6.0% | 6.0% |
| Citric acid (30% in water) | 1.5% | 1.5% | 1.5% | 1.5% |
| ANTIL ® 200 PEG-200 Hydrogenated glyceryl | 1.5% | 1.5% | 1.5% | 1.5% |
| palmate; PEG-7 glyceryl cocoate | | | | |
| Sodium chloride | 1% | 1% | 1% | 1% |
| Water | ad 100% | ad 100% | ad 100% | ad 100% |
| Evaluation: | | | | |
| Wet combability | 3.5 | 2.5 | 4.5 | 3.0 |
| Wet feel | 4.2 | 2.5 | 4.5 | 3.2 |
| Dry combability | 4.0 | 3.5 | 4.5 | 3.5 |
| Dry feel | 4.5 | 3.2 | 5.0 | 3.8 |

Compared to the comparison products C1 and C2, the hair shampoos 1 and 2 according to the invention each exhibit better conditioning properties. In particular, the evaluation of the feel of the wet hair and after drying comes out significantly better in the case of shampoos 1 and 2 containing the conditioner according to the invention as in example 4. The evaluations detailed in the table are made on the scale: 1=poor to 5=excellent. The assessment was made using sections of hair by an evaluation panel.

While the invention has been described herein with reference to specific embodiments, features and aspects, it will be recognized that the invention is not thus limited, but rather extends in utility to other modifications, variations, applications, and embodiments, and accordingly all such other modifications, variations, applications, and embodiments are to be regarded as being within the spirit and scope of the invention.

What is claimed is:

1. A method of producing organomodified siloxanes with domain-type distribution comprising, partially or completely reacting
A) hydrogensiloxanes with a degree of distribution (persistency ratio) (η) of components [A] and [B] in the copolymer [AB]
$$η = \frac{[ A ] [ B ]}{[ AB ]}$$
of η>1, with
B) olefinically unsaturated compounds, acetylenically unsaturated compounds or a mixture of said unsaturated compounds.

2. The method of producing organomodified siloxanes as claimed in claim 1, wherein, for producing methylsiloxanes, a mixture of hexamethyldisiloxane, poly(methyl)hydrogensiloxane and siloxane cycles are equilibrated as starting material.

3. The method of producing organomodified siloxanes as claimed in claim 1, wherein the olefinically unsaturated compounds of the general formula (1) CH₂═CHR—(CH₂)ₙ-A are used, in which A is at least one radical selected from the group of
1. optionally substituted linear and/or cyclic hydrocarbons which optionally contain functional groups,
2. randomly or blockwise constructed oxyalkylene chains —(OAlk)ₛ-R, in which
R is H, a C₁₋₁₈-alkyl radical which is optionally substituted and/or optionally contains heteroatoms, or the radical of a monobasic and/or polybasic C₁₋₂₂-carboxylic acid, which can optionally contain hydroxyl groups, the radical of an inorganic acid selected from the group sulfuric acid, sulfonic acid, phosphoric acid,
OAlk is the radical -(EO)_{b}—(PO)_{c}—(BO)_{d}-(DO)ₑ—(SO)_{f}— where
EO is ethylene oxide radical,
PO is propylene oxide radical,
BO is butylene oxide radical,
DO is dodecenyl oxide radical,
SO is styrene oxide radical,
b, c, d, e, f are ≧0 in particular
b is 0 to 50,
c is 0 to 50,
d, e, f are identical or different and are 0 to 10
and the sum
b+c+d+e+f is a and
a is 1 to 50,
and
3. radicals containing oxirane groups of the formulae 3a to 3d 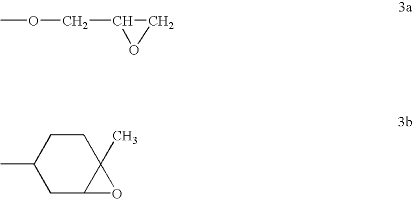 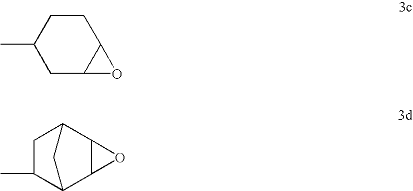
(and when x=0)
4. at least one amine/ammonium radical, -MZ, -[M-Z]^{g+}h*X^{j-}, in which
M is a divalent radical chosen from the group 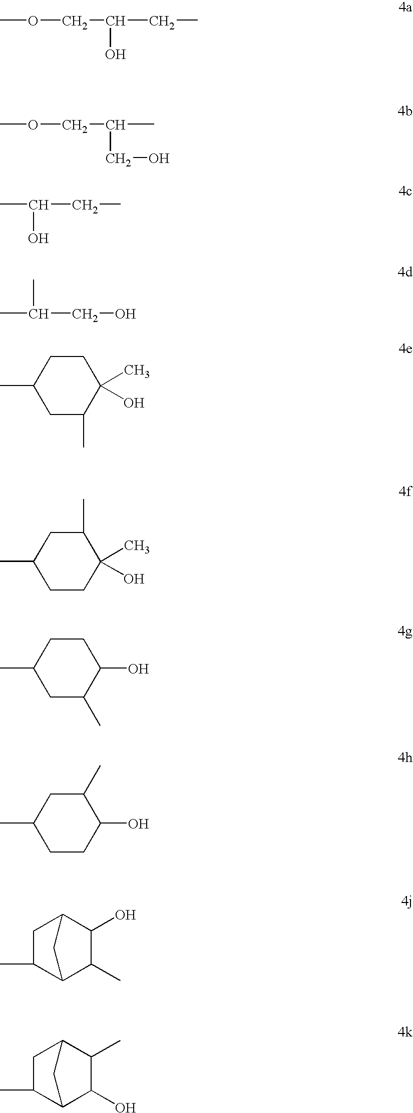
and Z may be one of the radicals 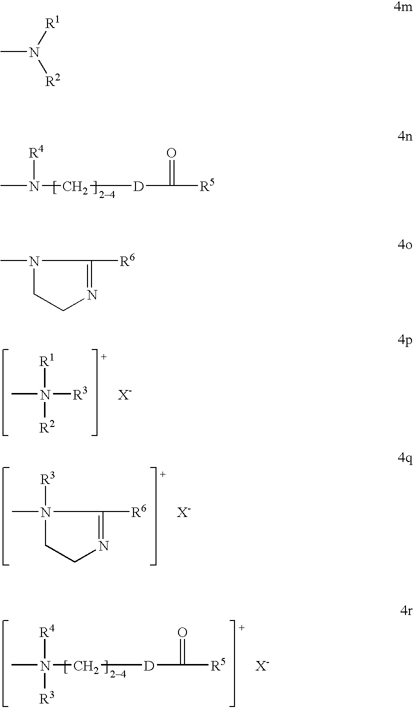
in which
R¹, R², R³, R⁴, independently of one another, are H, C₁₋₂₂-alkyl radicals which may also contain hydroxyl groups,
R⁵, R⁶ are C₁₋₂₂-alkyl radicals which may also contain hydroxyl groups,
D is —O— or —NR⁷— where R⁷=alkyl or hydroxyalkyl radical with 1-4 carbon atoms,
X⁻ may be an organic or inorganic anion,
h*j is the same as the numerical value of g,
5. polyhydroxyorganyl radicals of the general formula —R⁸—PH, where the radical
R⁸ acts as spacer between siloxane backbone and polyhydroxyorganyl radical or sugar radical and is of the type known from the prior art for polyhydroxyorganyl- or sugar-modified siloxanes,
PH is a polyhydroxyorganyl radical which contains a defined number n of (C—OH) groups, where n is ≧2, preferably 5 to 15,
from the group mono-, di-, oligo- or polysaccharide, their glycosides or corresponding derivatives, in particular glucose, maltose, raffinose, sorbitol, glucosamine, glucopyranosylamine, glucamine, N-methylglucamine, isomaltamine, gluconic acid, heptagluconic acid; 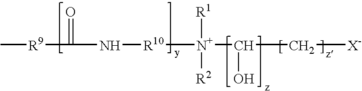
6. betaine groups,
in which
X is —COO—, SO₃⁻, PO₄²⁻,
R⁹ is alkylene radicals having up to 10 carbon atoms,
R¹⁰ is alkylene radicals having up to 2 to 6 carbon atoms,
y is 0 or 1,
Z is 0 or 1,
z′ is 1, 2 or 3,
and R¹, R² are identical or different and have the meaning given above, together form an imidazoline ring or are —CH₂—CH₂—OH, —(CH₂)_{z}—X⁻.
7. guanidine groups of the formulae (6a, 6b or 6c)
R¹¹=—N-G (6a)
R¹¹=—N-Q⁺A⁻ (6b)
R=—(N)ₓ—S (6c)
in which
G is a guanidino group with the general formula (6a′, 6a²) 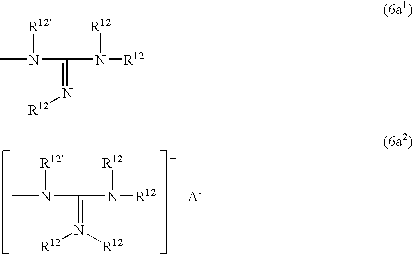
and/or salts or hydrates thereof, in which
R¹², independently of the others, is hydrogen or an optionally branched hydrocarbon radical optionally containing double bonds, or
R^{12′} may be R¹² or an alkylene group which is joined to M via carbon atoms or heteroatoms and thus forms a 5- to 8-membered ring and
N is a di- or polyvalent hydrocarbon radical having at least 4 carbon atoms which has a hydroxyl group and which may be interrupted by one or more oxygen atoms or nitrogen atoms or quaternary ammonium groups or esters or amide functions,
Q⁺ is a radical of the formula (6d) 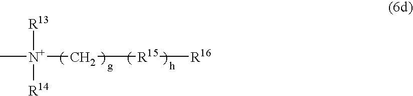
R¹³, R¹⁴ are alkyl radicals having 1 to 4 carbon atoms,
R¹⁵ is 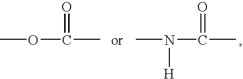
R¹⁶ may be a monovalent hydrocarbon radical having 1 to 22 carbon atoms,
g is 0 to 6
h is 0 or 1,
A⁻ is an inorganic or organic anion which originates from a customary physiologically compatible acid HA,
S is H, a polyalkylene oxide polyether of the general formula
CₘH₂ₘO(C₂H₄O)ₘ(C₃H₆O)ₒR⁷
in which
m is 1 to 6, in particular 3, 6,
n, o, independently of one another, are 0 to 100, in particular 0 to 20 and the polyether has a molecular weight between 100 and 6000 g/mol and
R¹⁷ is H or an optionally branched aromatic or alicyclic hydrocarbon radical having 2 to 30 carbon atoms, preferably 4 to 22 carbon atoms, and optionally containing double bonds, or a UV-absorbing group, in particular cinnamic acid or methoxycinnamic acid.

4. An organomodified siloxane with block character produced as claimed in accordance to claim 1.

5. A method for producing and stabilizing cosmetic formulations comprising adding at least one organomodified siloxane with a block character obtained in accordance to claim 1 to a cosmetic formulation in an amount effect to stabilize said cosmetic formulation.

6. The method as claimed in claim 5, wherein, for producing methylsiloxanes, a mixture of hexamethyldisiloxane, poly(methyl)hydrogensiloxane and siloxane cycles are equilibrated as starting material.

7. The method as claimed in claim 5, wherein the olefinically unsaturated compounds of the general formula (1) CH₂═CHR—(CH₂)ₙ-A are used, in which A is at least one radical selected from the group of
1. optionally substituted linear and/or cyclic hydrocarbons which optionally contain functional groups,
2. randomly or blockwise constructed oxyalkylene chains —(OAlk)ₛ-R, in which
R is H, a C₁₋₁₈-alkyl radical which is optionally substituted and/or optionally contains heteroatoms, or the radical of a monobasic and/or polybasic C₁₋₂₂-carboxylic acid, which can optionally contain hydroxyl groups, the radical of an inorganic acid selected from the group sulfuric acid, sulfonic acid, phosphoric acid,
OAlk is the radical -(EO)_{b}—(PO)_{c}—(BO)_{d}-(DO)ₑ—(SO)_{f}— where
EO is ethylene oxide radical,
PO is propylene oxide radical,
BO is butylene oxide radical,
DO is dodecenyl oxide radical,
SO is styrene oxide radical,
b, c, d, e, f are ≧0, in particular
b is to 50,
c is 0 to 50,
d, e, f are identical or different and are 0 to 10
and the sum
b+c+d+e+f is a and
a is 1 to 50,
and
3. radicals containing oxirane groups of the formulae 3a to 3d 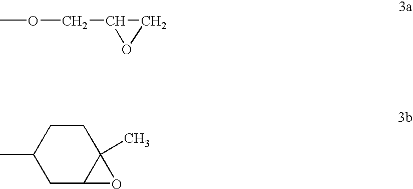
(and when x=0)
4. at least one amine/ammonium radical, -MZ, -[M-Z]^{g+}h*X^{j-}, in which
M is a divalent radical chosen from the group 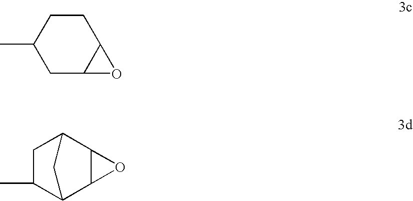
and Z may be one of the radicals 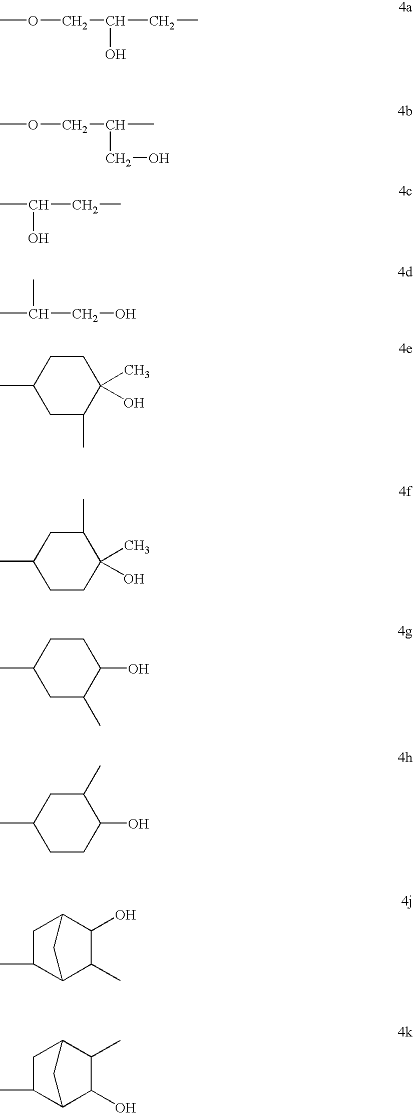 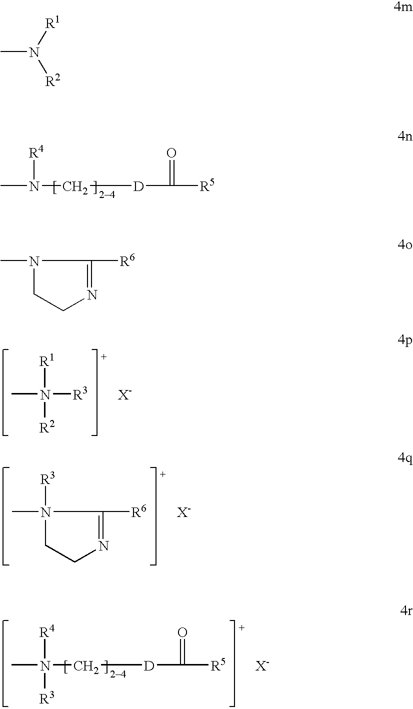
in which
R¹, R¹, R³, R⁴, independently of one another, are H, C₁₋₂₂-alkyl radicals which may also contain hydroxyl groups,
R⁵, R⁶ are C₁₋₂₂-alkyl radicals which may also contain hydroxyl groups,
D is —O— or —NR⁷— where R⁷=alkyl or hydroxyalkyl radical with 1-4 carbon atoms,
X⁻ may be an organic or inorganic anion,
h*j is the same as the numerical value of g,
5. polyhydroxyorganyl radicals of the general formula —R⁸—PH, where the radical
R⁸ acts as spacer between siloxane backbone and polyhydroxyorganyl radical or sugar radical and is of the type known from the prior art for polyhydroxyorganyl- or sugar-modified siloxanes,
PH is a polyhydroxyorganyl radical which contains a defined number n of (C—OH) groups, where n is ≧2, preferably 5 to 15,
from the group mono-, di-, oligo- or polysaccharide, their glycosides or corresponding derivatives, in particular glucose, maltose, raffinose, sorbitol, glucosamine, glucopyranosylamine, glucamine, N-methylglucamine, isomaltamine, gluconic acid, heptagluconic acid; 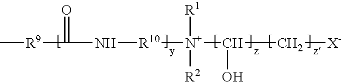
6. betaine groups,
in which
X is —COO—, SO₃⁻, PO₄²⁻,
R⁹ is alkylene radicals having up to 10 carbon atoms,
R¹⁰ is alkylene radicals having up to 2 to 6 carbon atoms,
y is 0 or 1,
Z is 0 or 1,
z′ is 1, 2 or 3,
and R¹, R² are identical or different and have the meaning given above, together form an imidazoline ring or are —CH₂—CH₂—OH, —(CH₂)ₙ—X⁻,
7. guanidine groups of the formulae (6a, 6b or 6c)
R¹¹=—N-G (6a)
R¹¹=—N-Q+A⁻ (6b)
R=—(N)ₓ—S (6c)
in which
G is a guanidino group with the general formula (6a′, 6a²) 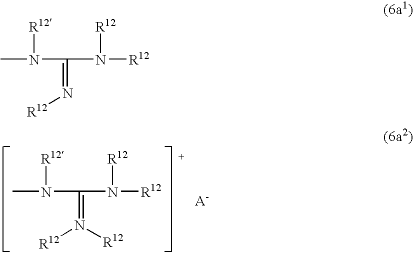
and/or salts or hydrates thereof, in which
R¹², independently of the others, is hydrogen or an optionally branched hydrocarbon radical optionally containing double bonds, or
R^{12′} may be R¹² or an alkylene group which is joined to M via carbon atoms or heteroatoms and thus forms a 5- to 8-membered ring and
N is a di- or polyvalent hydrocarbon radical having at least 4 carbon atoms which has a hydroxyl group and which may be interrupted by one or more oxygen atoms or nitrogen atoms or quaternary ammonium groups or esters or amide functions,
Q⁺ is a radical of the formula (6d) 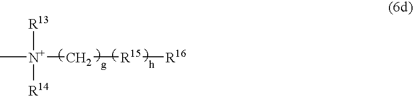
R¹³, R¹⁴ are alkyl radicals having 1 to 4 carbon atoms,
R¹⁵ is 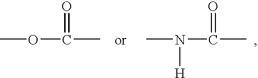
R¹⁶ may be a monovalent hydrocarbon radical having 1 to 22 carbon atoms, g is 0 to 6 h is 0 or 1,
A⁻ is an inorganic or organic anion which originates from a customary physiologically compatible acid HA,
S is H, a polyalkylene oxide polyether of the general formula
CₘH₂ₘO(C₂H₄O)ₙ(C₃H₆O)ₒR⁷
in which
m is 1 to 6, in particular 3, 6,
n, o, independently of one another, are 0 to 100, in particular 0 to 20 and the polyether has a molecular weight between 100 and 6000 g/mol and
R¹⁷ is H or an optionally branched aromatic or alicyclic hydrocarbon radical having 2 to 30 carbon atoms, preferably 4 to 22 carbon atoms, and optionally containing double bonds, or a UV-absorbing group, in particular cinnamic acid or methoxycinnamic acid.

8. A formulation which comprises one or more organomodified siloxanes with block character as claimed in claim 4.
